(19) United States
(12) Patent Application Publication
(10) Pub. No.: US 2002/0156509 A1
(43) Pub. Date: Oct. 24, 2002

(54) Thermal control suit

(76) Inventor: Stephen Cheung, Herring Cove (CA)

Correspondence Address: JOHN A. BAKER, KIRBY EADES GALE BAKER, BOX 3432, STATION D, OTTAWA, ON, K1P 6N9, CA

(21) Appl. No.: 10/126,659

(22) Filed: Apr. 22, 2002

Related U.S. Application Data
(60) Provisional application No. 60/285,232, filed on Apr. 23, 2001.

(51) Int. Cl. A61F 7/00; A61F 7/12

(52) U.S. Cl. 607/96; 607/108

(57) ABSTRACT

A portable thermal control module (TCM), for use in rapid heating or cooling the body, is provided. This thermal control module creates or displaces heat through the Peltier effect. A system and method of dynamically controlling the temperature of a body using multiple TCMs is also provided. This system and method involves a flexible thermal control suit (TCS) and a microprocessor.

### RELATED APPLICATION

This application is related to Provisional Patent Application Serial No. 60/285,232, filed Apr. 23, 2001.

### FIELD OF THE INVENTION

This invention relates to garments and devices to heat or cool human or animal subjects operating in environments involving thermal stress, or subjects for which thermal control is desired for medical, research, athletic conditioning, or environmental protection reasons.

### BACKGROUND OF THE INVENTION

The study of thermophysiology deals with the response of human and animal bodies to thermal stress. Such research has a wide range of applications including astronaut suits, North Atlantic oil rig worker clothing, and sport. Better understanding how to effectively apply heat and cooling to the human body will improve the protective clothing and thermal energy control regimes used in these and other temperature-challenged environments. For example, from such studies, clothing designers will know the most effective locations in a jacket to position additional insulation material; rescuers will know the most effective locations and methods to supply heat to hypothermia victims.

In the majority of thermophysiological studies, thermal stress has been imposed on the human or animal subject through exposure to a uniform ambient environment, such as that found during water immersion or in an environmental chamber.

An improved means for applying thermal stress to a human or animal body is known as liquid conditioned garments (LCGs). In thermophysiological research, LCGs essentially provide an individualized environmental chamber. There are two types of LCGs—active LCGs and passive LCGs. Active LCGs were developed by the National Aeronautics and Space Administration (NASA) for use by astronauts during extravehicular activities, and consist of an undergarment worn next to the skin with tubing stitched throughout. By running water through the tubing, heating or cooling of the astronaut is achieved.

Present active LCGs consist of water-perfused tubing stitched into a tight-fitting undergarment and worn next to the skin. The flowing water or fluid acts as the mechanism of heat exchange. Altering the rate and temperature of water flow through the tubing controls the rate of heat exchange. An external heater/cooler for the fluid is required, along with a water pump to circulate the water. If separate zones of thermal control are desired, a separate water pump and heater/cooler is required for each zone of control. A separate garment of tubing would also have to be manufactured to accommodate the change in thermal control.

There are several limitations to active LCGs. Since an LCG suit is designed for a particular body size, a suit may not be reusable. Achieving multiple zones of temperature control (e.g., arms, torso, legs), a desirable ability for thermophysiological research, would require a separate water source, pump, and temperature exchanger for each zone, greatly increasing complexity and cost. The most sophisticated models are presently capable of only three zones. Further limitations of the active LCG include uneven distribution of thermal stress over the body, no ability for dynamic temperature change, and limited ability for the subject to control the temperature himself.

An example of the prior art of active Liquid Conditioned Garments is U.S. Pat. No. 5,862,675, issued Jan. 26, 1999, to Scaringe et al. This particular design is a portable, vehicle mounted system utilizing traditional refrigeration-type, air-conditioning methods to pump cooled water through the garment.

Passive LCGs involve the placement of self-contained heat sources or cold sources adjacent to a human or animal body. At the Atlanta and Sydney Olympics, Australian rowers wore ice vests prior to competition to keep their body temperature from overheating. This is an example of a passive LCG.

Like active LCGs, passive LCGs have limitations for use in thermophysiological research. For example, no thermal control is possible in the rate of heat exchange. There is the risk of skin trauma (e.g., frostbite or burning). Since the rate of heat exchange decreases over time due to melting or diffusion, an additional cold source or heat source is required to continue heat transfer.

The present invention provides an improvement over the prior art, and provides a thermal control module, a thermal control suit for distributing the modules about the body, and a system and method for controlling the temperature of multiple modules. The invention employs commercially available thermoelectric modules (TEMs), which are devices making use of the Peltier effect. The Peltier effect is a phenomena whereby electric current, sent though a circuit made of dissimilar conducting materials, causes heat to be absorbed at one junction and given up at the other. Both TEMs and the Peltier effect are well known in the art.

Varying the direction and magnitude of current flow through the TEM controls the rate of heat exchange, causing one surface of the TEM to become cold and the opposite surface to become hot. Which surface becomes cold and which surface becomes hot is controlled by the direction of the current flowing through the device. The rate of heat transfer from one side of the TEM to the other, and therefore the degree of cold or heat, depends on the magnitude of the current. For example, if a skin surface is in direct or indirect contact with the hot side of the TEM, thermal energy will flow from the hot side of the TEM into the body.

The use of TEMs and the Peltier effect in an attempt to control body temperature is not new. U.S. Pat. No. 4,962,761, issued Oct. 16, 1990 to Golden further discloses a thermal bandage to be placed against the skin for heating and cooling. This bandage comprises a conforming member, a thermal pack, and an optional plate between the conforming member and the pack. This invention is limited as it provides no means of regulating and maintaining a thermal gradient across the thermal pack.

Although Golden also discloses “a thermal garment having a plurality of pockets into which ‘thermal bandages’ can be placed, he does not provide any method for dynamic temperature control over the various areas of the body, which practically limits the use of his suit.

### SUMMARY OF THE INVENTION

One aspect of the present invention involves individual thermal control modules (TCMs) consisting of a form-fitting, energy distributing pad of water, gel or other heat conducting fluid against the skin, an aluminum, copper or other heat conducting plate to maintain a solid surface between the pad and the TEM; a thermoelectric module (TEM) to affect heat exchange; and a heat sink to remove heat from the upper surface of the TEM in order to maintain a thermal gradient across the TEM.

Another aspect of the present invention is a multi-zone Thermal Control Suit (TCS) that is capable of manipulating and maintaining the internal body temperature of a human or an animal at regulated temperatures. The TCS consists of a number of TCMs, their controllers, a reconfigurable suit webbing, and a controlling computer or microprocessor

In accordance with one aspect of the present invention, there is provided a thermal control module for use in warming or cooling the surface of a subject, comprising: a form-fitting energy distributing pad; a thermoelectric module having an active surface and a reactive surface; and a heat sink in contact with said reactive surface of said thermoelectric module; where, when said thermal control module is warming said surface, said heat sink inputs thermal energy into said reactive surface and when said thermal control module is cooling said surface, said heat sink extracts heat energy from said reactive surface.

In accordance with another aspect of the present invention there is provided A system for independently controlling the temperature of specific zones of a body, comprising: one or more thermal control modules located in each of said zones in thermal contact with the body; a microprocessor associated with each of said zones for controlling and monitoring the temperature of the body within each of said zones; wherein said microprocessor compares said temperature with a predetermined set temperature to produce a signal for controlling operation of said one or more thermal control modules to thereby control the temperature of said one or more zones.

In accordance with still another aspect of the present invention there is provided a method of controlling a plurality of thermal control modules, comprising the steps of: operatively dividing said plurality of thermal control modules into one or more zones; associating each of said one or more zones to a desired temperature value; receiving a plurality of temperature signals from said plurality of thermal control modules; comparing each of said plurality of temperature signals to the desired temperature value associated with the corresponding zone; determining the appropriate amount and direction of electric current required to change the temperature of each of said plurality of thermal control modules to the desired temperature associated with the corresponding zone; and delivering said appropriate amount and direction of current to said plurality of thermal control modules.

In accordance with still another aspect of the present invention there is provided An adjustable webbing structure for wear on at least a portion of a subject, said webbing structure comprising: at least one flexible strap adjustably associated with one or more body parts of said subject; individual thermal control modules reconfigurably and removably mounted on said at least one strap; wherein each thermal control module contains a thermoelectric module.

### BRIEF DESCRIPTION OF THE DRAWINGS

The present invention will be discussed in detail by way of example using the following drawings, in which:

FIG. 1 shows the detailed structure of a particular embodiment of a Thermal Control Module (TCM). This particular embodiment is designed for continuous use and includes a water or fluid based heat sink on the outside surface of the TEM.

FIG. 2 is an embodiment of the suit showing one particular configuration of webbing to place a number of Thermal Control Modules (TCMs) on a human subject. Not shown are the zone controllers or central computer.

FIG. 3 shows the system by which the temperature of the Thermal Control Modules (TCMs) are dynamically controlled.

FIG. 4 is a sample of the prior art method based on Liquid Conditioned Garments (LCGs).

### DETAILED DESCRIPTION OF THE INVENTION

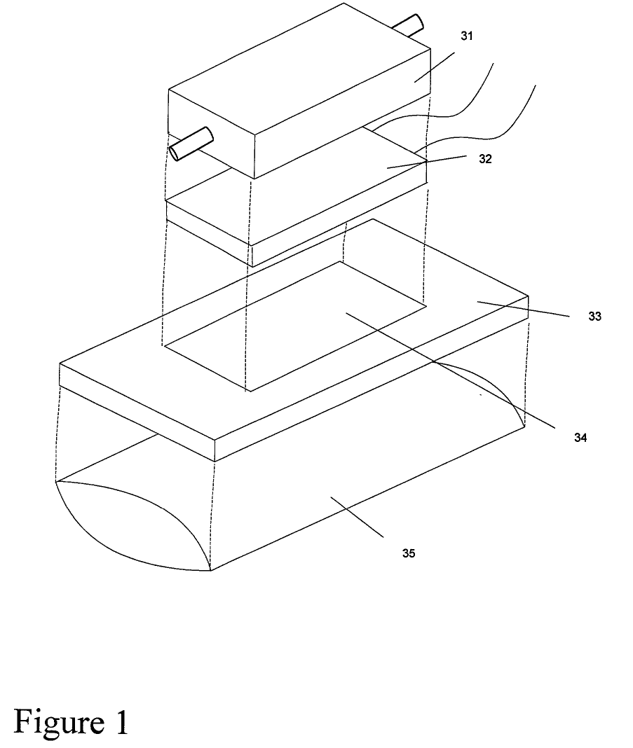

FIG. 1 shows an embodiment of a Thermal Control Module (TCM) of the present invention which is used to heat or cool a subject. The operation of this embodiment is described in terms of heating.

As already described, an individual TCM contains a thermoelectric module (TEM) 32 that causes the heat exchange. In order for the TEM 32 to continue to supply thermal energy to the subject body, two things must occur. Heat sink 35 is provided to act as a source of the thermal energy to be “pumped” into the subject and, and to maintain a thermal gradient across the TEM. This same function could be performed by another object, such as a metal heat radiator, a finned-type structure, a large capacity or phase change material based heat sink block, or in the heating mode, a simple electrical heating unit. It should be noted that unlike the prior art liquid conditioned garments, this heat sink merely provides a source or sink of thermal energy. When cooling the body, the heat sink functions in exactly the same manner but in the opposite direction by acting as a stable sink for heat energy “pumped” from the body by the TEM.

The TCM is placed on the body such that a liquid filled bag 35 is next to the skin. This bag is able to conform to the body surface and maintain the heat exchange surface between the skin and the TCM. The primary purpose of the pad is to spread heat exchange evenly throughout a relatively large surface area, rather than to maintain a focused source of heat next to the skin, as is the function of the majority of therapeutic heating/cooling pads. One appropriate substantiation of such a pad measures 4×4 inches and contains 2.5 fluid ounces of water. Aluminum plate 34 is optionally provided to maintain a solid surface between the bag and the TEM, encouraging heat transfer. Neoprene insulation 33, which covers the top of bag 35 and surrounds metal plate 34 and TEM 32, helps maintain the temperature of the fluid on bag 35 and presses the bag 35 closer to the skin. Neoprene insulation 33 is a preferred, but not a necessary part of this invention. In the TCM, there is no electrical current in contact with either water or the human body. The TEM operates at a maximum voltage of 15 V, which is far below that which would be harmful to the subject. The surface of the bag 35, the only part of the TCM that contacts the subject, is made from hypoallergenic plastic, and the risk of allergic reaction is negligible. FIG. 1 shows a fluid-based heat sink in contact with the reactive surface of the TEM. Some form of thermal sink is always necessary but it does not need to be the small, active, fluid-based structure shown. Dependent upon the specific experiment, application or large-scale thermal environment, fin-based or radiative structures can be used, fan based air can as a thermal sink, or even block-based heat sinks or phase change materials can be used.

Each individual thermal module is only capable of a maximum heat exchange of 20 W in the present embodiment. While this may cause mild heat or cold discomfort, it is not possible to sustain any thermal injuries (e.g. frostbite, burns) with this low amount of heat exchange. In addition, a localized 20W of heat from the TEM is diffused through the bag 35, further minimizing the localized effect of heat or cold.

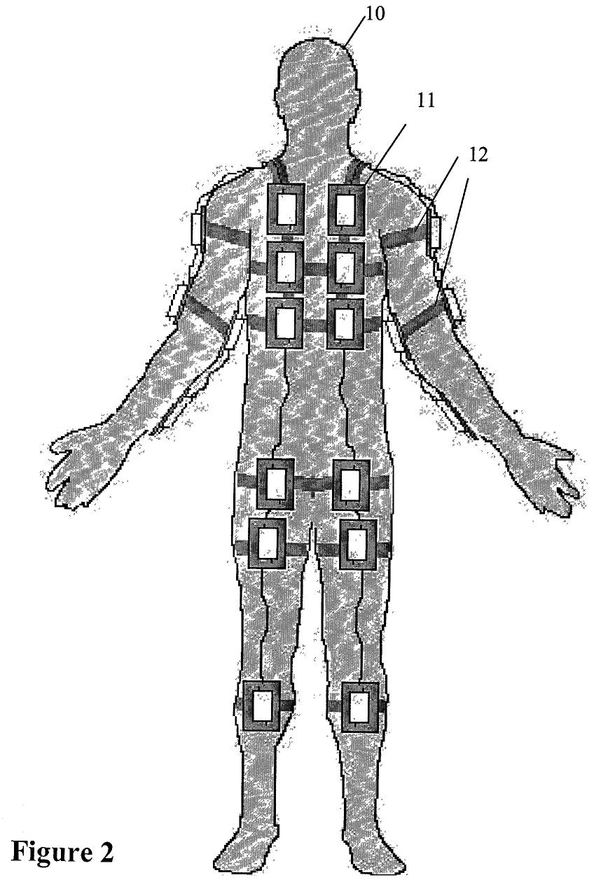

FIG. 2 shows an embodiment of a thermal control suit (TCS) of the present invention. The TCS is worn using a modular webbing system 12 that permits the flexible configuration of thermoelectric modules 11 throughout a body 10. Using this system, modules 11 may be concentrated in particular regions or specific areas of the body to maximize heat exchange or to accomplish specific physiological tasks. The modules 11 may be moved relatively quickly, and attach to the webbing system 12 using Velcro™ or the like. The use of a modular, reconfigurable webbing system 12 is very useful in a research environment, however it is within the scope of this application that TCMs covered by this application and their associated controllers and control mechanisms can also be mounted in full-cover garments, primarily for work environment uses. The preferred embodiment of a TCS permits the same suit to be used for a variety of heating or cooling regimens on a variety of different sized subjects.

Modules may also be added or removed from the TCS without affecting the heat exchange in other modules. It is not necessary to switch off or remove power from the suit or any portion thereof in order to add or remove TCMs as additional TCMs can be added and connected while the other TCMs are still under active control.

In one embodiment of the TCS, up to 40 TCMs can be accommodated on the body. Each of the 40 TCMs has a theoretical maximum rate of heat exchange (heating or cooling) of 20 W. Therefore, the maximum rate of heat exchange of this embodiment is 800 W. As a standard of reference, the average human at rest generates 100 W of heat calculated at a peak shivering heat production rate of 528 W. In this particular embodiment, up to 10 controllers are provided, each of which controls up to 4 TCMs.

It should also be appreciated that the thermal control suit covered by this application need not be a full body suit as shown in FIG. 2. Dependent upon the particular physiological purpose, the particular sports purpose or medical application, it may require only a partial suit, for example, upper torso, a single limb, the neck and armpit.

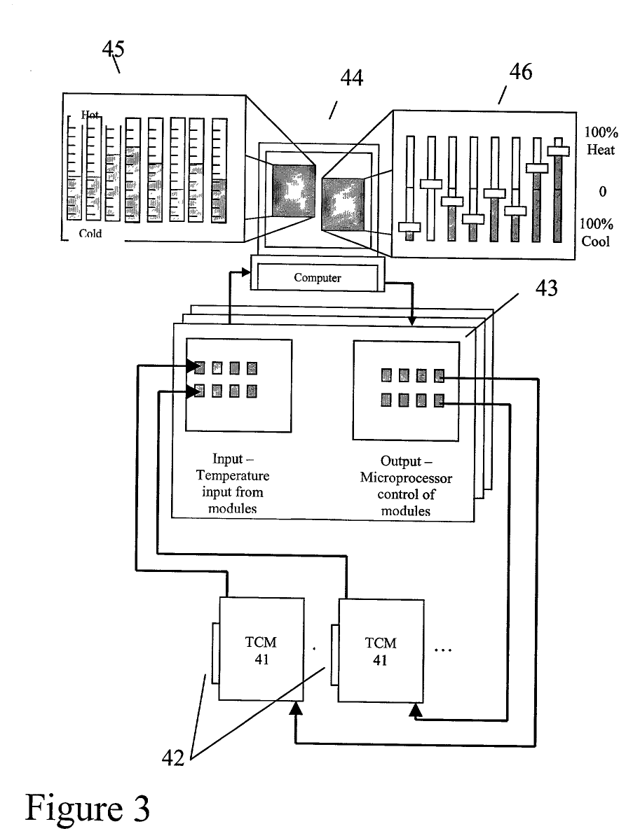

FIG. 3 shows a particular embodiment of the system used for monitoring and regulating the temperature throughout the TCS and the modules contained therein. Each TCM 41 has a temperature sensor 42 that detects the temperature of the skin underneath the module. The temperature of each TCM 41 is input into the corresponding zone controller 43, which contains a microprocessor. The temperature of each TCM 41 is sent to the computer 44 and is displayed graphically in the upper left of the computer screen 45. Each zone controller 43 then compares the temperatures of the TCMs 41 in its zone to a single pre-determined desired temperature for that zone and calculates whether cooling or heating for each TCM 41 is needed to achieve that desired temperature. The required degree of heating or cooling is displayed graphically in the upper right of the computer screen 46. The zone controller 43 then sends the appropriate direction and magnitude of current to each of the TCMs 41 in the zone. Alternative methods of control and communications between each zone controller and the TCMs include digital parallel communications from the computer to all zone controllers, zone controllers supplying TCMs in series configurations, and the monitoring of individual TCM temperature sensors by each zone controller and use of same for local distributed control and for return of values back to the central computer via the digital communications bus, and local microprocessor ability within the zone controllers for local temperature or thermal regime decision making.

The zone controllers 43, of which only one is shown in FIG. 3, contain the analog electrical components necessary to convert the control decisions of the computer and/or the microprocessor into the actual current flow rate and direction supplied to the TCMs 41. This current flow is shown in FIG. 3 as being supplied in parallel to two TCMs for the single zone controller shown. The TCMs within a given zone, under control of a single zone controller can be connected in series and supplied with current from a single supply line.

Although FIG. 3 illustrates thermal control based on skin temperature feedback from the TCMs, thermal control can also be achieved based on feedback from internal body temperature, heat flux, blood flow, or a combination of any of these parameters.

FIG. 3 shows a single zone. Other embodiments would provide a plurality of zones so that, for example, the torso could be defined on one zone and have a first desired temperature; the arms, another zone and have a second desired temperature, etc.

Several safety features can be incorporated as part of a preferred embodiment of this invention. The system can be designed to prevent both core body temperature and individual TEMs from moving beyond a particular range, for example, the range of 95° F.-105° F. for core body temperature and 35° F.-120° F. for individual TEMs. Should core body temperature reading move beyond this range, an alarm may flash on the computer and the TEMs may automatically be disabled. In addition, both the subject and the investigators may have access to separate large control buttons. Should either button be pressed, an alarm may flash on the computer and the TEMs may immediately be disabled.

A particular embodiment of the TCS is designed to be completely modular with up to 40 TEMs distributed in 1-10 zones of thermal control.

In one embodiment of the TCS, the modules, power source, heat sink, and control unit are sufficiently light and portable to permit individuals to move and work in a field setting. The TCS is therefore capable of being worn under any protective clothing and in different ambient environments.

Skin temperature can be dynamically controlled in each zone of the body, or across a number of zones, by the investigator or the subject. Body temperature can be regulated despite the ambient environment, despite the existing core body temperature, and despite changes in metabolic heat generation (e.g. those brought about by exercise or shivering).

This invention has been described involving skin temperature measurement. Another embodiment of the invention involves the measurement of core body temperature and controlling the zone temperatures according to an algorithm relating individual zone temperature to core body temperature.

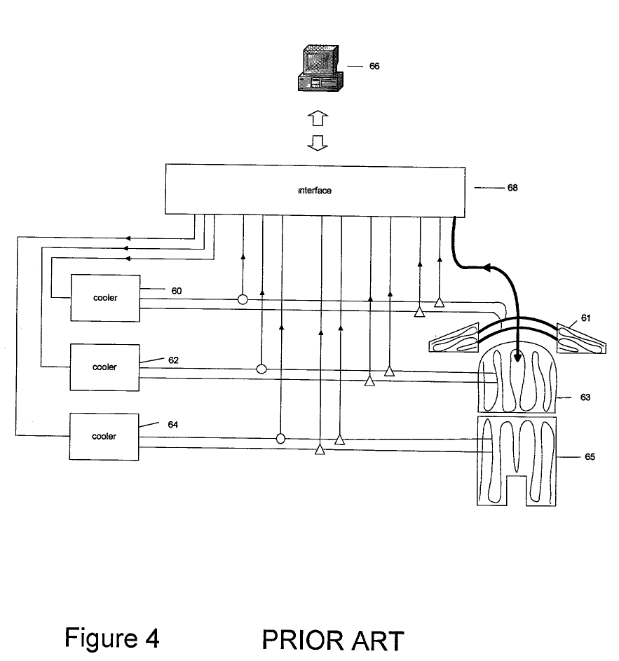

FIG. 4 shows an example of the prior art of liquid conditioned garments (LCGs). FIG. 4 shows three zones: 61, 63, and 65. Each zone is provided with a cooler 60, 62, and 64. Each cooler is controlled by a computer 66 via an interface 68. Each cooler includes a pump which pumps liquid conditioned by a controller through a zone.

1. A thermal control module for use in warming or cooling the surface of a subject, comprising:
a form-fitting energy distributing pad;
a thermoelectric module having an active surface and a reactive surface; and
a heat sink in contact with said reactive surface of said thermoelectric module;
where, when said thermal control module is warming said surface, said heat sink inputs thermal energy into said reactive surface and when said thermal control module is cooling said surface, said heat sink extracts heat energy from said reactive surface.

2. The thermal control module of claim 1, further comprising a heat conducting plate connecting said pad and said active surface of said thermoelectric module.

3. The thermal control module of claim 1, wherein said form-fitting energy distributing pad contains a heat conducting fluid.

4. The thermal control module of claim 1, further comprising an insulating means which covers an outer surface of said pad and surrounds said heat conductive plate and said thermoelectric module.

5. The thermal control module of claim 1 further comprising a temperature sensor for sensing the temperature of the surface of the subject directly beneath the thermal control module.

6. A system for independently controlling the temperature of specific zones of a body, comprising:
one or more thermal control modules located in each of said zones in thermal contact with the body;
a microprocessor associated with each of said zones for controlling and monitoring the temperature of the body within each of said zones; wherein said microprocessor compares said temperature with a predetermined set temperature to produce a signal for controlling operation of said one or more thermal control modules to thereby control the temperature of said one or more zones.

7. The system of claim 6 wherein each of said one or more thermal control modules comprises:
a form-fitting energy distributing pad;
a thermoelectric module having an active surface and a reactive surface; and
a heat sink in contact with said reactive surface of said thermoelectric module;
where, when said thermal control module is warming said surface, said heat sink inputs thermal energy into said reactive surface and when said thermal control module is cooling said surface, said heat sink extracts heat energy from said reactive surface.

8. A system for controlling core body temperature comprising:
a temperature sensor for determining core body temperature;
a plurality of thermal control modules in thermal contact with the body, wherein one or more of said thermal control modules are located in each of one or more zones of the body;
a microprocessor for independently controlling the body temperature within each of said zones;
wherein an algorithm associated with said microprocessor compares said core body temperature with a predetermined core body temperature to produce a signals for controlling the operation of said one or more thermal control modules to thereby control the core body temperature.

9. The system of claim 8 wherein each of said one or more thermal control modules comprises:
a form-fitting energy distributing pad;
a thermoelectric module having an active surface and a reactive surface; and
a heat sink in contact with said reactive surface of said thermoelectric module;
where, when said thermal control module is warming said surface, said heat sink inputs thermal energy into said reactive surface and when said thermal control module is cooling said surface, said heat sink extracts heat energy from said reactive surface.

10. A method of controlling a plurality of thermal control modules, comprising the steps of:
operatively dividing said plurality of thermal control modules into one or more zones;
associating each of said one or more zones to a desired temperature value;
receiving a plurality of temperature signals from said plurality of thermal control modules;
comparing each of said plurality of temperature signals to the desired temperature value associated with the corresponding zone;
determining the appropriate amount and direction of electric current required to change the temperature of each of said plurality of thermal control modules to the desired temperature associated with the corresponding zone; and
delivering said appropriate amount and direction of current to said plurality of thermal control modules.

11. An adjustable webbing structure for wear on at least a portion of a subject, said webbing structure comprising:
at least one flexible strap adjustably associated with one or more body parts of said subject;
individual thermal control modules reconfigurably and removably mounted on said at least one strap;
wherein each thermal control module contains a thermoelectric module.

12. The adjustable webbing structure of claim 11 wherein each thermal control module belongs to a particular physical zone of the subject body.

13. The adjustable webbing structure of claim 12 further comprising a microprocessor for controlling the temperature of each of said zones.
